(19) United States
(12) Patent Application Publication
(10) Pub. No.: US 2024/0115747 A1
(43) Pub. Date: Apr. 11, 2024

(54) Disinfecting Mat Device

(71) Applicant: James Galloway, Memphis, TN (US)

(72) Inventor: James Galloway, Memphis, TN (US)

(21) Appl. No.: 18/481,486

(22) Filed: Oct. 5, 2023

Related U.S. Application Data
(60) Provisional application No. 63/414,933, filed on Oct. 11, 2022.

(51) Int. Cl. A61L 2/10; A61L 2/18; A61L 2/24

(52) U.S. Cl. CPC A61L 2/10; A61L 2/18; A61L 2/24; A61L 2202/14; A61L 2202/15; A61L 2202/20

(57) ABSTRACT

A disinfecting mat device is provided. The device is comprised of a body with at least one, but preferably a plurality of layers with at least one UV light, at least one storage tank that stores at least one disinfecting agent, and at least one sensor. The device is designed to be used as a door mat, such that the device sanitizes the bottom of the shoes of the user as they step on the device by expelling sanitizing solution through the top surface. The device also simultaneously sanitizes the bottom of a user's shoes via the UV light when the user steps on the device.

### CROSS-REFERENCE TO RELATED APPLICATION

The present application claims priority to, and the benefit of, U.S. Provisional Application No. 63/414,933, which was filed on Oct. 11, 2022, and is incorporated herein by reference in its entirety.

### FIELD OF THE INVENTION

The present invention relates generally to the field of doormats. More specifically, the present invention relates to a disinfecting mat device that can be used as a doormat, wherein the device expels a disinfecting compound when stepped on and uses at least one UV light to disinfect the bottom of a user's shoes simultaneously. Accordingly, the present disclosure makes specific reference thereto. Nonetheless, it is to be appreciated that aspects of the present invention are also equally applicable to other like applications, devices, and methods of manufacture.

### BACKGROUND

Individuals often track germs, viruses, and bacteria into a home or businesses via their shoes. This causes contamination and may put others at risk of contracting illnesses. More specifically, many contaminants are brought into a home or business on the bottoms of shoes, which a regular doormat is not equipped to disinfect and sanitize.

Therefore, there exists a long-felt need in the art for an improved doormat device. There also exists a long-felt need in the art for a disinfecting mat device. More specifically, there exists a long-felt need in the art for a disinfecting mat device that disinfects the bottom of a user's shoes before entering a home, a business, etc.

The subject matter disclosed and claimed herein, in one embodiment thereof, comprises a disinfecting mat device. The device is comprised of a body with at least one, but preferably a plurality of layers with at least one UV light, at least one storage tank that stores at least one disinfecting agent, and at least one sensor. The device is designed to be used as a door mat, wherein the device sanitizes the bottom of the shoes of the user as they step on the device by expelling sanitizing solution through the top surface. The device also simultaneously sanitizes the bottom of a user's shoes via the UV light when the user steps on the device.

In this manner, the disinfecting mat device of the present invention accomplishes all the foregoing objectives and provides an improved doormat device. During use, the device disinfects the bottom of a user's shoes before entering a home, a business, etc. As a result, the device prevents germs, bacteria, and viruses from being tracked into a home, business, etc., via the shoes of the user.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a disinfecting mat device. The device is comprised of a body with at least one, but preferably a plurality of layers with at least one UV light, at least one storage tank that stores at least one disinfecting agent, and at least one sensor. The device is designed to be used as a door mat, wherein the device sanitizes the bottom of the shoes of the user as they step on the device before entering a home, a business, etc.

In the preferred embodiment, the body is comprised of a bottom layer, a middle layer, and a top layer. The bottom layer contacts the ground surface the device rests on during use. The bottom layer is preferably made from a non-slip rubber material to prevent the device from moving/slipping during use. The middle layer is preferably comprised of at least one UV light. The light may be of any wavelength that disinfects/kills germs, bacteria, viruses, etc. The light is powered by at least one battery that may be removable or rechargeable.

The light is activated to illuminate by at least one sensor which is preferably comprised of a pressure sensor. In this manner, the sensor detects when a user steps onto the body. The sensor may be comprised of a threshold weight (i.e., 40 pounds) that must be exceeded to allow the sensor to activate the light. This prevents the UV light from being wrongly activated by packages placed on the body, by pets, etc.

The top layer of the device may be comprised of at least one outline that may be raised, recessed, or flush. The outline is preferably the outline of two feet, wherein the UV lights are preferably positioned underneath the outlines in the middle layer. In this manner, the lights are concentrated on the soles of a user's shoes when the user places their shoes within the outline. The top layer is also comprised of at least one opening that allows at least one disinfecting agent to be expelled from at least one opening in the middle layer, through the opening of the top layer, and out of the top layer when a user steps on the body. In one embodiment, the opening is positioned within the shoe outline to ensure the disinfecting agent is concentrated on the soles of a user's shoes when the user places their shoes within the outline.

The present invention is also comprised of a method of using the device. First, a device is provided comprised of a body with at least one layer with at least one UV light, at least one storage tank that stores at least one disinfecting agent, and at least one sensor. Then, the disinfecting agent is poured within the storage tank such that it flows into the middle layer via the tubing. Next, a user steps onto the top layer (preferably into the shoe outline in an embodiment of the top layer with said outline) such that the sensor is activated to illuminate the UV light and the agent is excreted through the opening of the top layer. Then, the user steps off of the device and into a home, business, etc.

The present invention is also comprised of a second method of using the device. First, a device is provided comprised of a body with at least one layer with at least one UV light, at least one storage tank that stores at least one disinfecting agent, and at least one sensor. Then, the disinfecting agent is poured within the storage tank such that it flows into the middle layer via the tubing. Next, a user steps on or presses the button to activate the UV light. Then, a user steps onto the top layer (preferably into the shoe outline in an embodiment of the top layer with said outline) such that the sensor is activated to illuminate the UV light and the agent is excreted through the opening of the top layer. Then, the user steps off of the device and into a home, business, etc.

Accordingly, the disinfecting mat device of the present invention is particularly advantageous as it provides an improved doormat device. During use, the device disinfects the bottom of a user's shoes before entering a home, a business, etc. As a result, the device prevents germs, bacteria, and viruses from being tracked into a home, business, etc., via the shoes of the user. In this manner, the disinfecting mat device overcomes the limitations of existing doormats known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a disinfecting mat device of the present invention in accordance with the disclosed architecture;

FIG. 2 illustrates a cross-section side view of one potential embodiment of a disinfecting mat device of the present invention in accordance with the disclosed architecture;

FIG. 3 illustrates a flowchart of a method of using one potential embodiment of a disinfecting mat device of the present invention in accordance with the disclosed architecture; and

FIG. 4 illustrates a flowchart of a method of using one potential embodiment of a disinfecting mat device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved doormat device. There also exists a long-felt need in the art for a disinfecting mat device. More specifically, there exists a long-felt need in the art for a disinfecting mat device that disinfects the bottom of a user's shoes before entering a home, business, etc.

The present invention, in one exemplary embodiment, is comprised of a disinfecting mat device comprised of a body with at least one, but preferably a plurality of layers with at least one UV light, at least one storage tank that stores at least one disinfecting agent, and at least one sensor. The device is designed to be used as a doormat. During use, the device sanitizes the bottom of the shoes of the user as they step on the device before entering a home, business, etc.

In the preferred embodiment, the body is comprised of a bottom layer, a middle layer, and a top layer. The bottom layer contacts the ground surface the device rests on during use. The bottom layer is preferably made from a non-slip rubber material to prevent the device from moving/slipping during use. The middle layer is preferably comprised of at least one UV light of any wavelength that disinfects/kills germs, bacteria, viruses, etc. The light is powered by at least one battery which may be a removable or a rechargeable battery.

The light is activated to illuminate by at least one sensor, which is preferably comprised of a pressure sensor. In this manner, the sensor detects when a user steps onto the body. The sensor may be comprised of a threshold weight (i.e., 40 pounds) that must be exceeded to allow the sensor to activate the light. This prevents the UV light from being wrongly activated by packages placed on the body, by pets, etc.

The top layer of the device may be comprised of at least one outline. The outline may be raised, recessed, or flush in different embodiments. The outline is preferably the outline of two feet, wherein the UV lights are preferably positioned underneath the outlines in the middle layer. In this manner, the lights are concentrated on the soles of a user's shoes when the user places their shoes within the outline. The top layer is also comprised of at least one opening that allows at least one disinfecting agent to be expelled from at least one opening in the middle layer, through the opening of the top layer, and out of the top layer when a user steps on the body. In one embodiment, the opening is positioned within the shoe outline to ensure the disinfecting agent is concentrated on the soles of a user's shoes when the user places their shoes within the outline.

The present invention is also comprised of a method of using the device. First, a device is provided comprised of a body with at least one layer with at least one UV light, at least one storage tank that stores at least one disinfecting agent, and at least one sensor. Then, the disinfecting agent is poured within the storage tank, such that it flows into the middle layer via the tubing. Next, a user steps onto the top layer (preferably into the shoe outline in an embodiment of the top layer with said outline) such that the sensor is activated to illuminate the UV light and the agent is excreted through the opening of the top layer. Then, the user steps off of the device and into a home, business, etc.

The present invention is also comprised of a second method of using the device. First, a device is provided comprised of a body with at least one layer with at least one UV light, at least one storage tank that stores at least one disinfecting agent, and at least one sensor. Then, the disinfecting agent is poured within the storage tank such that it flows into the middle layer via the tubing. Next, a user steps on or presses the button to activate the UV light. Then, a user steps onto the top layer (preferably into the shoe outline in an embodiment of the top layer with said outline) such that the sensor is activated to illuminate the UV light and the agent is excreted through the opening of the top layer. Then, the user steps off of the device and into a home, business, etc.

Accordingly, the disinfecting mat device of the present invention is particularly advantageous as it provides an improved doormat device. During use, the device disinfects the bottom of a user's shoes before entering a home, business, etc. As a result, the device prevents germs, bacteria, and viruses from being tracked into a home, business, etc., via the shoes of the user. In this manner, the disinfecting mat device overcomes the limitations of existing doormats known in the art.

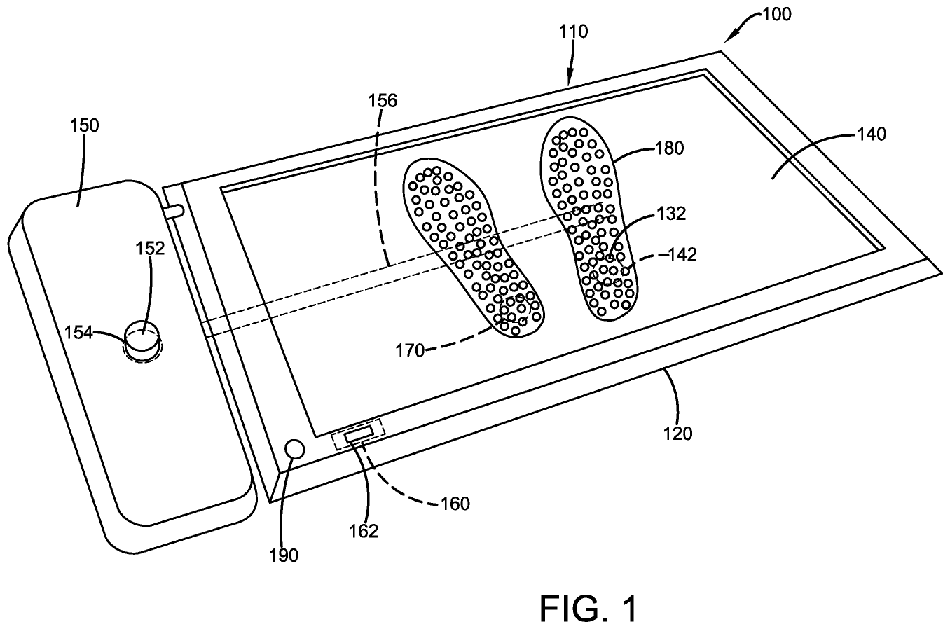

Referring initially to the drawings, FIG. 1 illustrates a perspective view of one potential embodiment of a disinfecting mat device 100 of the present invention in accordance with the disclosed architecture. The device 100 is comprised of a body 110 with at least one, but preferably a plurality of layers 120,130,140 with at least one UV light 132, at least one storage tank 150 that stores at least one disinfecting agent 158, and at least one sensor 170. The device 100 is designed to be used as a door mat, wherein the device 100 sanitizes the bottom of the shoes of the user as they step on the device 100 before entering a home, business, etc.

In the preferred embodiment, the body 110 is comprised of a bottom layer 120, a middle layer 130, and a top layer 140. The bottom layer 120 contacts the ground surface the device 100 rests on during use. The bottom layer 120 is preferably made from a non-slip rubber material to prevent the device 100 from moving/slipping during use. The bottom layer 120 may be textured with a raised or a recessed texture to further enhance the grip of the bottom layer 120 while resting on a ground surface.

The middle layer 130 is preferably comprised of at least one UV light 132. The UV light 132 may be of any type in the art. The light 132 may be of any wavelength that disinfects/kills germs, bacteria, viruses, etc. The light 132 is powered by at least one battery 160 that may be a removable or a rechargeable battery. In one embodiment, the battery 160 can be recharged via a USB charging port 162 (or other charging port of the like). The light 132 is activated to illuminate by at least one sensor 170. The sensor 170 is preferably comprised of a pressure sensor. In this manner, the sensor 170 detects when a user steps onto the body 110. The sensor 170 may be comprised of a threshold weight (i.e., 40 pounds) that must be exceeded to allow the sensor 170 to activate the light 132. This prevents the UV light 132 from being wrongly activated by packages placed on the body 110, by pets, etc. In one embodiment, the light 132 can be turned on/off via at least one button 190 that may be pressed or stepped on by the user in lieu of, or in addition to, the sensor 170. In one embodiment, the light 132 automatically turns off after a period of time after the light 132 is illuminated by the button 190. In a further embodiment, the light 132 may continuously illuminate.

The top layer 140 of the device 100 may be comprised of at least one outline 180 that may be raised, recessed, or flush. The outline 180 is preferably the outline of two feet. The UV lights 132 are preferably positioned underneath the outlines 180 in the middle layer 130. In this manner, the lights 132 are concentrated on the soles of a user's shoes when the user places their shoes within the outline 180.

In one embodiment, the top layer 142 is comprised of at least one opening 142. The opening 142 allows at least one disinfecting agent 158 to be expelled from at least one opening 134 (which extends to the top layer 140 opening 142) in the middle layer 130, through the opening 142, and out of the top layer 140 when a user steps on the body 110. In one embodiment, the opening 142 is positioned within the shoe outline 180 to ensure the disinfecting agent 158 is concentrated on the soles of a user's shoes when the user places their shoes within the outline 180. To prevent overspill of the agent 158, the shoe outline 180 may be recessed in the top layer 140 in one embodiment.

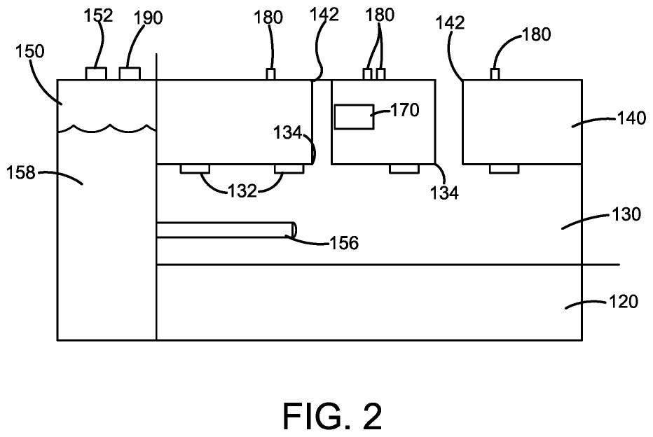

The disinfecting agent 158 may be any disinfecting, anti-bacterial, anti-viral, sanitizing, etc., compound known in the art which can disinfect the soles of a user's shoes. The agent 158 is stored within at least one stored tank 150. The tank 150 is in fluid communication with the middle layer 130 via at least one tubing 156, as seen in FIG. 2. The agent 158 can be poured into the tank 150 via at least one opening 154 covered by at least one cap 152. The cap 152 and opening 154 are preferably threaded.

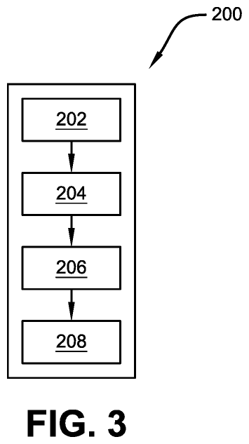

The present invention is also comprised of a method of using 200 the device 100, as seen in FIG. 3. First, a device 100 is provided comprised of a body 110 with at least one layer 120,130,140 with at least one UV light 132, at least one storage tank 150 that stores at least one disinfecting agent 158, and at least one sensor 170 [Step 202]. Then, the disinfecting agent is poured within the storage tank 150 such that it flows into the middle layer 130 via the tubing 156 [Step 204]. Next, a user steps onto the top layer 140 (preferably into the shoe outline 170 in an embodiment of the top layer 140 with said outline 170) such that the sensor 170 is activated to illuminate the UV light 132 and the agent 158 is excreted through the opening 142 of the top layer 140 [Step 206]. Then, the user steps off of the device 100 and into a home, business, etc. [Step 208].

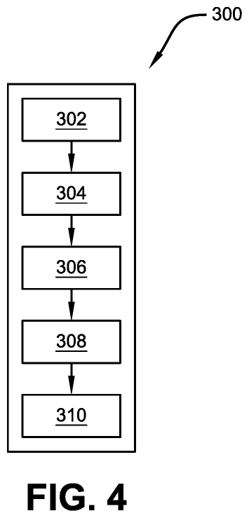

The present invention is also comprised of a second method of using 300 the device 100, as seen in FIG. 4. First, a device 100 is provided comprised of a body 110 with at least one layer 120,130,140 with at least one UV light 132, at least one storage tank 150 that stores at least one disinfecting agent 158, and at least one sensor 170 [Step 302]. Then, the disinfecting agent is poured within the storage tank 150, such that it flows into the middle layer 130 via the tubing 156 [Step 304]. Next, a user steps on or presses the button 190 to activate the UV light 132 [Step 306]. Then, a user steps onto the top layer 140 (preferably into the shoe outline 170 in an embodiment of the top layer 140 with said outline 170) such that the sensor 170 is activated to illuminate the UV light 132 and the agent 158 is excreted through the opening 142 of the top layer 140 [Step 308]. Then, the user steps off of the device 100 and into a home, business, etc. [Step 310].

Certain terms are used throughout the following description and claim to refer to particular features or components. As one skilled in the art will appreciate, different persons, may refer to the same feature or component by different names. This document does not intend to distinguish between components or features that differ in name but not structure or function. As used herein “disinfecting mat device” and “device” are interchangeable and refer to the disinfecting mat device 100 of the present invention.

Notwithstanding the foregoing, the disinfecting mat device 100 of the present invention and its various components can be of any suitable size and configuration as is known in the art without affecting the overall concept of the invention, provided that they accomplish the above-stated objectives. One of ordinary skill in the art will appreciate the size, configuration, and material of the disinfecting mat device 100 as shown in the FIGS. are for illustrative purposes only, and many other sizes and shapes of the disinfecting mat device 100 are well within the scope of the present disclosure. Although the dimensions of the disinfecting mat device 100 are important design parameters for user convenience, the disinfecting mat device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfecting mat device comprising:
a top layer comprised of an opening;
a middle layer comprised of a UV light and a tubing;
a bottom layer;
a battery;
a sensor;
a storage tank in fluid communication with the tubing; and
a disinfecting solution.

2. The disinfecting mat device of claim 1, wherein the bottom layer is comprised of a non-slip material.

3. The disinfecting mat device of claim 1, wherein the sensor is comprised of a pressure sensor.

4. The disinfecting mat device of claim 1, wherein the disinfecting solution is comprised of a disinfecting solution, an anti-bacterial solution, an anti-viral solution, or a sanitizing solution.

5. The disinfecting mat device of claim 1, wherein the top layer is comprised of an outline.

6. The disinfecting mat device of claim 5, wherein the outline is comprised of a foot outline.

7. The disinfecting mat device of claim 5, wherein the outline is raised or recessed.

8. The disinfecting mat device of claim 5, wherein the UV light is positioned under the outline.

9. The disinfecting mat device of claim 1, wherein the sensor activates the UV light.

10. The disinfecting mat device of claim 1, wherein the middle layer is comprised of an opening.

11. A disinfecting mat device comprising:
a top layer comprised of an opening;
a UV light;
a tubing;
a bottom layer;
a battery;
a button;
a storage tank in fluid communication with the tubing; and
a disinfecting solution.

12. The disinfecting mat device of claim 11, wherein the button activates the UV light.

13. The disinfecting mat device of claim 11, wherein the top layer is comprised of an outline.

14. The disinfecting mat device of claim 13, wherein the outline is comprised of a foot outline.

15. The disinfecting mat device of claim 13, wherein the outline is raised or recessed.

16. The disinfecting mat device of claim 13, wherein the UV light is positioned under the outline.

17. The disinfecting mat device of claim 13, wherein the sensor is comprised of a pressure sensor.

18. The disinfecting mat device of claim 17, wherein the sensor is comprised of a threshold activation weight.

19. A method of using a disinfecting mat device, the method comprising:
providing a disinfecting mat device comprised of a body comprised of a top layer, a middle layer, and a bottom layer, a UV light, a storage tank that stores a disinfecting agent, and a sensor;
pouring the disinfecting agent into the storage tank such that it flows into the middle layer via a tubing; and
stepping onto the top layer such that the sensor is activated to illuminate the UV light and such that the disinfecting agent is excreted through an opening of the top layer.

20. The method of claim 19 further comprised of a step of pushing a button to activate the UV light.
